(19) United States
(12) Patent Application Publication
(10) Pub. No.: US 2026/0174989 A1
(43) Pub. Date: Jun. 25, 2026

(54) BLADDER SIMULATION DEVICE, KIT THEREFOR, AND RELATED METHOD

(71) Applicant: REAL LIFE SIMULATION CONSULTANTS, Barrie (CA)

(72) Inventors: Molly BECKER, Barrie (CA); Earla Simpson, Wasaga Beach (CA)

(21) Appl. No.: 19/430,534

(22) Filed: Dec. 23, 2025

Related U.S. Application Data
(60) Provisional application No. 63/737,956, filed on Dec. 23, 2024.

(51) Int. Cl. A61M 25/00; A61M 25/10

(52) U.S. Cl. CPC A61M 25/0017; A61M 25/1002; A61M 25/10184; A61M 2025/1086

(57) ABSTRACT

A bladder simulation device comprises a one-way valve and a contractible reservoir fluidically connected at one end of the valve. The valve is arranged to permit flow into the reservoir but to block flow out thereof. Thus, the fluid is releasable upon bypassing the valve, for example, using a catheter, to permit the fluid to evacuate by contraction of an elastic peripheral wall of the reservoir. A kit including the simulation device also comprises a syringe for refilling the reservoir which is preferably resiliently elastic. A method of simulating a bladder includes steps of providing such a simulation device and inserting the catheter to bypass the valve as to release the fluid in the reservoir by contraction of its peripheral wall.

This application claims the benefit under 35 U.S.C. 119(e) of U.S. provisional application Ser. No. 63/737,956 filed Dec. 23, 2024.

### TECHNICAL FIELD

The following relates to a bladder simulation device.

### BACKGROUND

Simulation is widely used in healthcare education to help students and professionals develop the necessary skills and competencies needed to work as safe practitioners. A skill frequently taught in nursing, medical and midwifery schools and hospitals is urinary catheterization. For educational purposes, learning this skill involves following the required steps and inserting a catheter into a simulated urinary tract, which may be provided on a mannequin. Ideally, simulated urine flows from an artificial bladder through the simulated urinary tract to mimic real life. Learners need this immediate and accurate feedback that mimics real life events to truly learn to properly care for their patient populations.

The artificial bladders typically used on mannequins are made of rigid plastics material that is difficult to seal due to their inflexibility. This causes fluid (simulated urine) to leak inside the mannequin which requires the mannequin to be dried and is generally inconvenient.

The artificial bladders are typically a solid shape (fixed shape) reservoir that can be filled with fluid, for example, water to simulate urine. The flow of the fluid from the bladder is controlled by a one-way check valve. When a catheter is inserted, the tip of the catheter opens the valve but only the water that comes in contact with the tip of the catheter empties (like a straw in a cup). To overcome this, the artificial (simulated) bladder often needs to be manipulated to change the orientation of the reservoir to increase the fluid evacuation. This is of course unrealistic in the real world and renders the artificial bladder ineffective for real world simulation.

An alternative form of task trainer uses a large pressurized system to help the fluid empty from the reservoir. These systems require a source of pressurized air, and therefore more set-up and maintenance, and cannot fit into an abdomen or full-body mannequin for immersive simulations.

Most bladders need to be emptied and cleaned after each use, an unrealistic task without human resources in place.

### SUMMARY

It is an object of the present invention to obviate or mitigate the above disadvantages.

According to an aspect of the invention, there is provided a bladder simulation device comprising:
a one-way valve having opposite first and second ends and defining therebetween a fluidic passageway, wherein the one way valve comprises at least one gate in the fluidic passageway and positionable in an open position in which the first and second ends are fluidically intercommunicated and a closed position in which the first and second ends are fluidically obstructed, wherein the one-way valve is configured to permit flow of a fluid in a first direction from the first end towards the second end responsive to a positive type of fluidic pressure differential therebetween and to resist said flow in a second direction from the second end towards the first end responsive to a negative type of the fluidic pressure differential therebetween, wherein the first end is connectable to a support structure for fluidic communication with a hole therein; and
a contractible reservoir fluidically connected to the second end of the one-way valve for storing the fluid, wherein the contractible reservoir is formed by an elastic peripheral wall configured to contract so as to release the fluid when the at least one gate is arranged in the open position.

This arrangement can evacuate the fluid without reliance on gravity.

Preferably, the peripheral wall of the reservoir is resiliently elastic as to be stretchable and contractible.

In the illustrated arrangement, the contractible reservoir is in the form of a balloon.

In the illustrated arrangement, the bladder simulation device further comprises an external clamp for sealingly connecting the contractible reservoir over the second end of the one-way valve.

In the illustrated arrangement, the bladder simulation device further comprises a coupler supported on the first end of the one-way valve for connecting to the support structure. 2

In the illustrated arrangement, the coupler comprises a threaded nut rotatably supported on the one-way valve.

In the illustrated arrangement, the at least one gate of the one-way valve comprises a pair of gates arranged in series.

In the illustrated arrangement, an upstream one of the gates relative to the flow of fluid in the first direction is in the form of a diaphragm configured to conform to an object passed through the upstream gate to bypass the upstream gate.

According to another aspect of the invention, there is provided a kit for simulating a bladder, comprising:
a device for simulating the bladder, wherein the device comprises:
   a one-way valve having opposite first and second ends and defining therebetween a fluidic passageway, wherein the one way valve comprises at least one gate in the fluidic passageway and positionable in an open position in which the first and second ends are fluidically intercommunicated and a closed position in which the first and second ends are fluidically obstructed, wherein the one-way valve is configured to permit flow of a fluid in a first direction from the first end towards the second end responsive to a positive type of fluidic pressure differential therebetween and to resist said flow in a second direction from the second end towards the first end responsive to a negative type of the fluidic pressure differential therebetween, wherein the first end is connectable to a support structure for fluidic communication with a hole therein; and
   a contractible reservoir fluidically connected to the second end of the one-way valve for storing the fluid, wherein the contractible reservoir is formed by a resiliently elastic peripheral wall configured to contract so as to release the fluid when the at least one gate is arranged in the open position; and
a syringe for refilling the device, wherein the syringe is operatively fluidically connectable with the contractible reservoir through the one-way valve to bypass said at least one gate for injecting fluid into the contractible reservoir.

This provides an arrangement for a reusable bladder simulation device.

Preferably, said at least one gate comprises a pair of gates arranged in series.

In the illustrated arrangement, an upstream one of the gates relative to the flow of fluid in the first direction is in the form of a diaphragm configured to conform to an object passed through the upstream gate to bypass the upstream gate.

In the illustrated arrangement, the kit further comprises a catheter configured to be fluidically connected to the syringe and to pass through the one-way valve in a manner as to bypass said at least one gate.

According to yet another aspect of the invention, there is provided for simulating catheterization of a bladder, comprising:
providing a simulated bladder comprising a one-way valve and a contractible reservoir containing fluid; and
inserting a catheter into the simulated bladder as to bypass the one-way valve and permit the contractible reservoir to release the fluid therefrom by contraction of the reservoir.

This provides an arrangement for simulating catheterization in which fluid flows out of the simulated bladder, via the catheter, substantially independently of gravity.

In the illustrated arrangement, the method further includes filling the simulated bladder with the fluid using a syringe fluidically connected with the contractible reservoir in a manner as to bypass the one-way valve.

In the illustrated arrangement, the syringe is fluidically connected with the contractible reservoir using the catheter passed through and bypassing the one-way valve.

In one such arrangement, the method further comprises, after filing the simulated bladder with the fluid using the syringe, removing the catheter from the one-way valve.

When the one-way valve comprises a pair of series gates, and a first one of the series gates upstream relative to a permitted flow of fluid through the one-way valve is in the form of a diaphragm, preferably filling the simulated bladder using the syringe comprises simultaneously bypassing both of the series gates while sealingly engaged by the upstream gate to resist leakage of the fluid out of the simulated bladder.

Filling the simulated bladder using the syringe may be performed while the simulated bladder is connected to a support structure.

In the illustrated arrangement, removing the catheter from the one-way valve to stop the release of the fluid from the reservoir.

These and other aspects are contemplated and described herein. It will be appreciated that the foregoing summary sets out representative aspects of the invention to assist skilled readers in understanding the following detailed description.

### DESCRIPTION OF THE DRAWINGS

A greater understanding of the embodiments will be had with reference to the Figures, in which:

FIG. 1 is a schematic sectional view of an arrangement of bladder simulation device according to the present invention, connected to a support structure with a catheter bypassing a one-way valve of the device;

FIG. 2 is a schematic sectional view of an arrangement of kit for simulating a bladder comprising the bladder simulation device of FIG. 1 and a syringe;

FIG. 3 is a cross-sectional view along line 3-3 in FIG. 1;

FIG. 4 is a schematic representation of a portion of a support structure for use with an arrangement of bladder simulation device according to the present invention; and

FIG. 5 is an enlarged view of the arrangement of bladder simulation device of FIG. 4.

### DETAILED DESCRIPTION

For simplicity and clarity of illustration, where considered appropriate, reference numerals may be repeated among the Figures to indicate corresponding or analogous elements. In addition, numerous specific details are set forth in order to provide a thorough understanding of the embodiments described herein. However, it will be understood by those of ordinary skill in the art that the embodiments described herein may be practised without these specific details. In other instances, well-known methods, procedures and components have not been described in detail so as not to obscure the embodiments described herein. Also, the description is not to be considered as limiting the scope of the embodiments described herein.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender. Further definitions for terms may be set out herein; these may apply to prior and subsequent instances of those terms, as will be understood from a reading of the present description.

The accompanying figures show a bladder simulation device indicated at 100, which is particularly suited for simulating bladder catheterization. The device 100 comprises a one-way valve 102 having opposite first and second ends 104, 105 and defining therebetween a fluidic passageway 108. That is, the valve 102 comprises a tubular body 109 defining the first and second ends 104, 105 and forming the passageway 108 through which fluid can flow between the opposite ends 104, 105. The valve 102 further comprises at least one gate 112 in the fluidic passageway 108, that is, in the body 109 and between the ends 104, 105, for controlling flow of fluid therethrough. The at least one gate 112 disposed in the passageway is positionable in an open position in which the first and second ends 104, 105 are fluidically intercommunicated and a closed position in which the first and second ends 104, 105 are fluidically obstructed. When there are plural gates, for example two gates 112A and 112B, typically each of the gates is positioned in a respective open position to permit flow fluid past the respective gate, such that the opposite valve ends are fluidically intercommunicated, and to fluidically obstruct the passageway 108, at least one of the gates is positioned in a respective closed position in which the respective gate resists flow of the fluid past the same as to substantially fluidically seal or close the passageway. Further, the one-way valve 102 is configured to permit flow of the fluid in a first direction D1 from the first valve end 104 towards the second valve end 105 responsive to a positive type of fluidic pressure differential therebetween and to resist the flow in an opposite second direction D2, that is, from the second end 105 towards the first end 104, responsive to a negative type of the fluidic pressure differential between the first end and the second end. This may be provided by design of the at least one gate such that it is conduced to the open position responsive to a pressure differential across a respective one of the at least one gate which is positive, and conduced to the closed position responsive to a pressure differential across the respective gate that is negative. Further, the first valve end 104 is connectable to a support structure 10 for fluidic communication with a hole 12 therein. Preferably, the support structure 10 forms a substantially enclosed container, such that the bladder simulation device 100 is not visible.

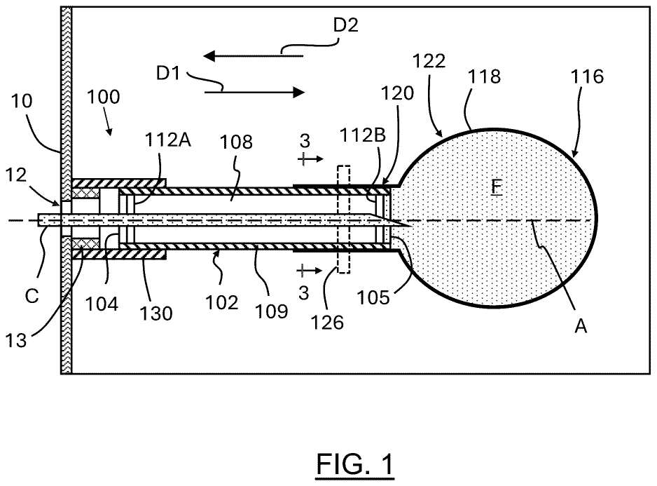

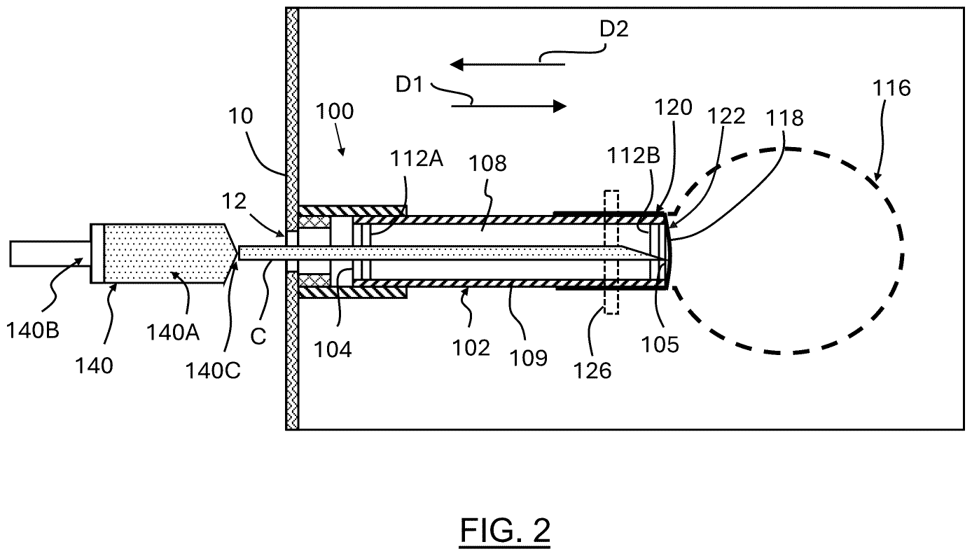

Additionally, the simulation device 100 comprises a contractible reservoir 116 fluidically connected to the second valve end 105 for storing fluid F. The contractible reservoir 116 is formed by an elastic peripheral wall 118 configured to contract so as to release the fluid when the at least one gate 112 is arranged in the open position. That is, the reservoir peripheral wall 118 is configured to be tensioned when containing fluid F, so that the wall 118 is conduced to contract when the pressure in the reservoir can be relieved, which action will cause the fluid to evacuate the reservoir. As such, the reservoir 116 is arrangeable in a filled condition in which the reservoir contains simulation fluid, in this case a liquid simulating urine, in which state the reservoir is conduced to contract as to evacuate the simulation fluid contained therein, as shown, for example, in FIG. 1 (solid line) or in phantom in FIG. 2, and an empty condition in which the reservoir cannot contract (any further) and there is substantially no simulation fluid contained therein, as shown, for example, in FIG. 2. The bladder simulation device 100 is thus configured to release or evacuate the fluid, when the one-way valve 102 is opened, without reliance on gravity.

As such, it will be appreciated that the hole 12 in the support structure and fluidic passageway 108 of the valve are collectively generally representative of a urinary tract and the reservoir 116 is representative of a bladder.

In the illustrated embodiment, the contractible reservoir 116 is in the form of a balloon. That is, the contractible reservoir comprises a neck 120 and a body 122 collectively formed by the elastic peripheral wall 118, such that the neck and body are unitary. The neck 120 is received over the second valve end 105 and supported by the valve body 109, such that the balloon body 122 is cantileverly supported therefrom (particularly in the filled condition). Preferably, the peripheral wall 118 has a prescribed thickness (between an interior surface exposed to the fluid F and an exterior surface opposite thereto, which faces the support structure) such that the reservoir body 122 is supported in substantially centered relation to the valve, as shown by substantially coaxial relation of the body 122 to axis A of axially-extending valve body 109.

When the reservoir has a valve-mounting portion which is received over the valve body, as in the case of the neck 120, the device 100 further includes an external clamp 126 for sealingly connecting the contractible reservoir over the second end of the one-way valve. For example, the clamp 126 comprises an adjustable collar formed, for example, by a releasable fastener, such as a zip-tie.

To mount the simulation device 100 to the support structure 10, the simulation device 100 preferably includes a coupler 130 supported on the first valve end 104. That is, the coupler 130 is distinct from the valve body 109, and in the illustrated embodiment, comprises a threaded nut rotatably supported on the one-way valve, that is, on the valve body 109. In such an arrangement, the nut may be threadably coupled to an externally threaded stem 13 which is carried on the support structure 10 in registered relation with the hole 12.

In the illustrated embodiment, the peripheral wall 118 of the reservoir is resiliently elastic as to be stretchable and contractible. That is, the wall 118 is movable between the filled and empty conditions of the reservoir. As such, the bladder simulation device can be refilled as to be reusable.

To facilitate reuse, the device 100 is provided as a kit with a syringe 140 for refilling the bladder 100. In the illustrated arrangement, the syringe comprises a barrel 140A for containing simulation fluid and a plunger 140B movably supported by the barrel to expel the fluid therefrom. The barrel 140B includes a tip or hub 140C from which the fluid is releasable from the barrel. The syringe (schematically shown) is operatively fluidically connectable with the contractible reservoir through the one-way valve 102 to bypass the gate(s) for injecting fluid into the reservoir 116. In the illustrated arrangement, the syringe is operatively fluidically connected with the reservoir using the catheter C, which is configured to be fluidically connected to the syringe and to pass through the one-way valve in a manner as to bypass its gate(s). Thus, the catheter C may be provided with the kit, too.

To reduce leakage during refilling, the illustrated embodiment of simulation device 100 the at least one gate 112 comprises a pair of gates 112A, 112B arranged in series. That is, the one-way valve comprises a pair of gates in a common fluidic passageway. In the illustrated arrangement, each of the gates are disposed substantially at a corresponding one of the ends or 105 of the valve 102, and there is no intervening element within the passageway between the gates, such that they are adjacent one another within the fluidic passageway.

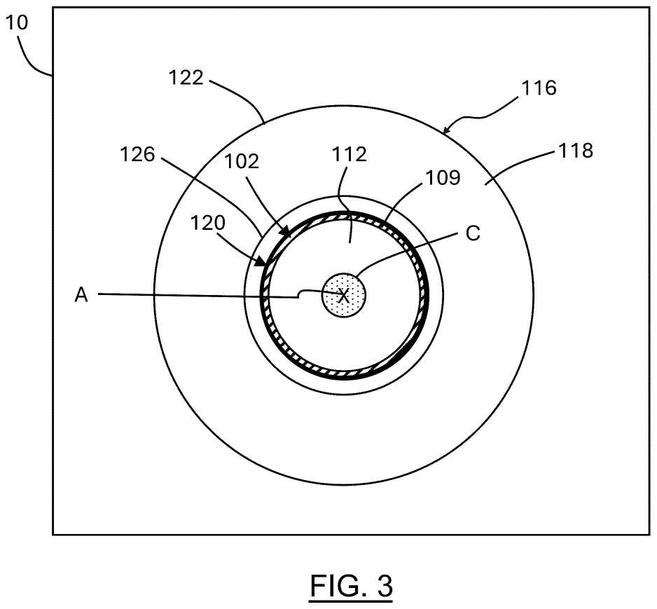

To enhance leakage protection, an upstream one of the gates relative to the flow of fluid in the first direction D1, in this case 112A, is in the form of a diaphragm configured to conform to an object, such as a catheter, passed through the upstream gate to bypass the upstream gate, as shown, for example, in FIG. 3. The diaphragm type gate comprises a deformable sheet spanning diametrically across the fluidic passageway 108 with a central location or spot that can be enlarged into an opening by stretching. As such, if any fluid bypasses a downstream one of the gates relative to fluid flow in the first direction, in this case 112B, the partially closed upstream gate 112A resists the fluid flow past the same and out of the valve. In the illustrated embodiment, both the upstream and downstream gates are of the diaphragm type.

There is also disclosed herein a method for simulating catheterization of a bladder. The method generally comprises:
providing a simulated bladder 100 comprising a one-way valve 102 and a contractible reservoir 116 containing fluid F; and
inserting a catheter C into the simulated bladder 100 as to bypass the one-way valve 102 and permit the contractible reservoir 116 to release the fluid F therefrom by contraction of the reservoir. 16

The catheter C, which forms a tube, provides a path for the fluid F to flow (in the second direction D2) whereby the fluid bypasses one or more control gates 112 of the one-way valve. Thus, a pressure applied on the fluid by the peripheral wall 118 of the reservoir which is conduced to contract is no longer opposed by the one-way valve 102, so the fluid can flow out of the reservoir and more generally out of the bladder simulation device. The reservoir 116 acts to release the fluid F so long as the catheter C is bypassing the one-way valve 102 such that there is a path for the fluid to flow out of the reservoir.

Accordingly, the method of simulation preferably further includes removing the catheter C from the one-way valve 102 to stop the release of the fluid from the reservoir 116. If the catheter C is removed before all the fluid in the reservoir has evacuated, the one-way valve 102 will act to retain a remaining fluid in the reservoir 116, which can be expelled by contraction thereof when the catheter C is reinserted in a manner bypassing the valve 102.

Preferably, the method for simulating catheterization further comprises filling the simulated bladder 100 with the fluid using a syringe 140 fluidically connected with the reservoir 116 in a manner as to bypass the one-way valve 102. In the illustrated arrangement, the syringe is fluidically connected with the contractible reservoir 116 using catheter C, which is passed through and bypasses the one-way valve. Thus, the bladder device is reusable for multiple simulated catheterization procedures. During filling, fluid F flows in the first direction D1 into the reservoir 116. Preferably, the gate(s) 112 act to retain the fluid in the reservoir 116 during filling with the syringe.

After filling the simulated bladder with fluid using the syringe, the method of simulation includes removing the catheter from the one-way valve 102, such that fluid is retained in the reservoir 116. It will be appreciated that although the catheter C is usable both to release the fluid from the reservoir as well as to introduce or admit the fluid from a storage or transfer vessel, like a syringe, typically the catheter C is removed after being used to release the fluid and is reinserted to bypass the one-way valve when it is desired to refill the bladder. The catheter May be connected to the syringe prior to being inserted to the one-way valve as to resist release of remaining simulation fluid in the reservoir during refilling.

When the bladder's one-way valve comprises a pair of series gates 112A and 112B, and a first gate 112A upstream relative to a permitted flow of fluid through the one-way valve is in the form of a diaphragm, the step of filling the simulated bladder using the syringe 140 comprises simultaneously bypassing both of the series gates while sealingly engaged by (at least) the upstream gate 112A to resist leakage of the fluid out of the simulated bladder 100.

Filling the simulated bladder using the syringe 140 may be performed while the simulated bladder is connected to the support structure 10, such as a mannequin or enclosed container. That is, the syringe can be passed through the hole 12 in the support structure, and into a position bypassing the one-way valve, as to inject the fluid into the reservoir 116.

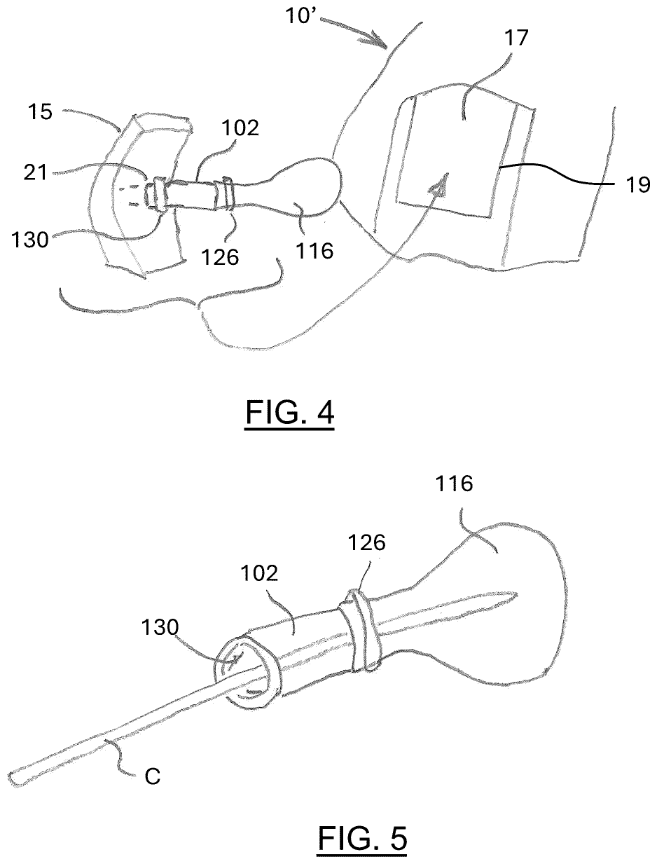

When the support structure 10 comprises a mannequin 10′ used for training of medical practitioners, as schematically shown in FIG. 4, the mannequin 10′ includes replica genitalia 15, either male or female. The mannequin 10′ has a void 17 to simulate a body cavity and an opening 19 to receive the genitalia 15.

The genitalia 15 have a urinary tract 21 that opens on one side of the genitalia 15 and extends through the genitalia 15 so it projects into the body cavity 17. A check valve 102 is attached to the urinary tract 21 by a screw thread 130. The check valve 102 allows fluid from outside the mannequin 10′ into the void 17 but inhibits flow from the cavity 17 to the outside of the mannequin body. 2

A contractible reservoir 116 in the form of a balloon is secured to the end of the urinary tract 18, which is generally formed by the check valve 102. The balloon 116 is made from an elastic flexible material, preferably a natural rubber or silicone, that allows the volume of the balloon 22 to expand as fluid is introduced.

Preferably, the free body dimensions of the balloon include 5.7 cm diameter and can expand to a volume of 0.5 litres.

The balloon 116 is secured to the check valve 102 by a releasable fastener 126 such as a zip tie to provide a fluid tight seal to the check valve 102.

In use, the ballon 116 is secured to the check valve 102 and the genitalia 15 inserted into the opening 19 so the balloon is within the body cavity 17.

An irrigation syringe 140 filled with the fluid F, that is, simulated urine, for example, water, is then inserted into the urinary tract 21 and the water transferred through the tract 21 to the balloon 116. The check valve 102 opens to allow the flow of fluid and the balloon expands to accommodate the water transferred. Typically, the syringe transfers 250 ml of water although more can be transferred if desired to expand the balloon further.

The syringe 140 is removed and the check valve 102 closes to prevent flow of fluid from the balloon 116. The expansion of the balloon 116 exerts a compressive force on the contents of the balloon 116 to expel the fluid if the check valve 102 is opened.

To simulate a urinary tract catheterization, a catheter C is inserted into the urinary tract 21 and moved along the tract until the tip of the catheter C encounters the check valve 102. The check valve 102 is forced open (upon contact with the catheter) and the fluid is discharged through the valve 102 and catheter C. Typically, the fluid flow is permitted until all the fluid is discharged, and the catheter 26 can then be withdrawn.

The bladder may then be refilled using the syringe 140 and a further practice session undertaken.

The simulated balloon bladder device 100 is a convenient and realistic way to practice urinary catheterization in the simulation setting. Using either the male or female genitalia that comes with the simulator, a one-way valve is connected with a removable balloon secured to the opposite end. The balloon 116 acts as the simulated urinary bladder. The genitalia can be reinserted at the bottom of the abdomen of the simulator in use.

The bladder remains hidden from participant view. Educators can quickly and easily fill the bladder with simulated urine using a syringe and urinary catheter. The balloon expands to account for the simulated urine and the one-way valve prevents any leakage.

When used in a simulation experience, participants can insert a urinary catheter into the urethral opening, passing through the one-way valve, allowing the bladder to empty as in real life. Due to the pressure inside the balloon from filling with simulated urine, the bladder is able to empty completely and with clinical accuracy without needing to manipulate the position of the simulator.

The balloon bladder can be used multiple times and quickly and easily changed if desired.

An example sequence employed in the use of the mannequin 10 is set out below:

### For Patient Catheterization—Assembly:

1. Assemble the Balloon Bladder by stretching the opening of the expandable balloon reservoir over the smooth end of the one-way valve, secure with a zip tie fastener.
2. Secure the one-way valve apparatus to the simulated genitalia using the screw cap connector on the one-way valve.
3. Insert the catheterization assembly into the perineal cavity of the patient simulator.

### For Patient Catheterization—Use:

1. Insert the patient catheterization tube through the one-way valve into the fluid filled expandable balloon reservoir. Simulated urine will begin to flow immediately on contact with the patient catheterization tube.
2. Fill the Balloon Bladder with the desired amount of water (can be coloured to simulate urine) using a standard Foley catheter and 60 cc syringe.

The foregoing arrangement is advantageous because:
1. The device can be used in a simulation scenario or skills lab.
2. Educators, i.e., users who assemble and/or fill the simulation device with fluid for subsequent evacuation, can quickly and easily fill the balloon before and between learners, using the balloon bladder repeatedly without needing to disassemble the device from the support structure, such as the mannequin and its genitalia.
3. The device can be reused without removal, cleaning and drying between uses, that is, after each complete evacuation of the simulated urine. 8
4. The reservoir and clamping device therefor can be quickly and easily changed, such as by following the afore-described assembly instructions.

As described hereinbefore, the present invention relates to a bladder simulation device which comprises a one-way valve and a contractible reservoir fluidically connected at one end of the valve. The valve is arranged to permit flow into the reservoir but to block flow out thereof.

Thus, the fluid is releasable upon bypassing the valve, for example, using a catheter, to permit the fluid to evacuate by contraction of an elastic peripheral wall of the reservoir. A kit including the simulation device also comprises a syringe for refilling the reservoir which is preferably resiliently elastic. A method of simulating a bladder includes steps of providing such a simulation device and inserting the catheter to bypass the valve as to release the fluid in the reservoir by contraction of its peripheral wall.

As described hereinbefore, in an aspect, the present invention provides a support structure, for example, a mannequin having simulated human genitalia including a urinary tract, or a simple enclosed container, and a check valve connected to the urinary tract. A bladder is connected to the urinary tract through the check vale. The bladder is formed as a balloon made from a flexible elastic material that can expand to increase the volume of the bladder and exert a compressive force on the contents of the bladder.

In the preferred embodiment, fluid, typically water, can be introduced into the bladder from a syringe connected to the urinary tract to inflate the bladder. The check valve seals the bladder. A catheter can then be inserted in the urinary tract to open the check valve and allow the egress of water.

While the aspects of the present embodiments are illustrated and described as having a certain arrangement of aspects and features, it is understood that any suitable arrangement can be used that retains the functions described with respect to the present embodiments.

Although the foregoing has been described with reference to certain specific embodiments, various modifications thereto will be apparent to those skilled in the art without departing from the spirit and scope of the invention as outlined in the appended claims.

1. A bladder simulation device comprising:
a one-way valve having opposite first and second ends and defining therebetween a fluidic passageway, wherein the one way valve comprises at least one gate in the fluidic passageway and positionable in an open position in which the first and second ends are fluidically intercommunicated and a closed position in which the first and second ends are fluidically obstructed, wherein the one-way valve is configured to permit flow of a fluid in a first direction from the first end towards the second end responsive to a positive type of fluidic pressure differential therebetween and to resist said flow in a second direction from the second end towards the first end responsive to a negative type of the fluidic pressure differential therebetween, wherein the first end is connectable to a support structure for fluidic communication with a hole therein; and
a contractible reservoir fluidically connected to the second end of the one-way valve for storing the fluid, wherein the contractible reservoir is formed by an elastic peripheral wall configured to contract so as to release the fluid when the at least one gate is arranged in the open position.

2. The bladder simulation device of claim 1 wherein the peripheral wall of the reservoir is resiliently elastic as to be stretchable and contractible.

3. The bladder simulation device of claim 1 wherein the contractible reservoir is in the form of a balloon.

4. The bladder simulation device of claim 1 further including an external clamp for sealingly connecting the contractible reservoir over the second end of the one-way valve.

5. The bladder simulation device of claim 1 further including a coupler supported on the first end of the one-way valve for connecting to the support structure.

6. The bladder simulation device of claim 5 wherein the coupler comprises a threaded nut rotatably supported on the one-way valve.

7. The bladder simulation device of claim 1 wherein the at least one gate of the one-way valve comprises a pair of gates arranged in series.

8. The bladder simulation device of claim 7 wherein an upstream one of the gates relative to the flow of fluid in the first direction is in the form of a diaphragm configured to conform to an object passed through the upstream gate to bypass the upstream gate.

9. A kit for simulating a bladder, comprising:
a device for simulating the bladder, wherein the device comprises:
a one-way valve having opposite first and second ends and defining therebetween a fluidic passageway, wherein the one way valve comprises at least one gate in the fluidic passageway and positionable in an open position in which the first and second ends are fluidically intercommunicated and a closed position in which the first and second ends are fluidically obstructed, wherein the one-way valve is configured to permit flow of a fluid in a first direction from the first end towards the second end responsive to a positive type of fluidic pressure differential therebetween and to resist said flow in a second direction from the second end towards the first end responsive to a negative type of the fluidic pressure differential therebetween, wherein the first end is connectable to a support structure for fluidic communication with a hole therein; and
a contractible reservoir fluidically connected to the second end of the one-way valve for storing the fluid, wherein the contractible reservoir is formed by a resiliently elastic peripheral wall configured to contract so as to release the fluid when the at least one gate is arranged in the open position; and
a syringe for refilling the device, wherein the syringe is operatively fluidically connectable with the contractible reservoir through the one-way valve to bypass said at least one gate for injecting fluid into the contractible reservoir.

10. The kit of claim 9 wherein said at least one gate comprises a pair of gates arranged in series.

11. The kit of claim 10 wherein an upstream one of the gates relative to the flow of fluid in the first direction is in the form of a diaphragm configured to conform to an object passed through the upstream gate to bypass the upstream gate.

12. The kit of claim 9 further comprising a catheter configured to be fluidically connected to the syringe and to pass through the one-way valve in a manner as to bypass said at least one gate.

13. A method for simulating catheterization of a bladder, comprising:
providing a simulated bladder comprising a one-way valve and a contractible reservoir containing fluid; and
inserting a catheter into the simulated bladder as to bypass the one-way valve and permit the contractible reservoir to release the fluid therefrom by contraction of the reservoir.

14. The method of claim 13 further comprising filling the simulated bladder with the fluid using a syringe fluidically connected with the contractible reservoir in a manner as to bypass the one-way valve.

15. The method of claim 14 wherein the syringe is fluidically connected with the contractible reservoir using the catheter passed through and bypassing the one-way valve.

16. The method of claim 15 further comprising after filing the simulated bladder with the fluid using the syringe, removing the catheter from the one-way valve.

17. The method of claim 14 wherein, when the one-way valve comprises a pair of series gates, wherein a first one of the series gates upstream relative to a permitted flow of fluid through the one-way valve is in the form of a diaphragm, filling the simulated bladder using the syringe comprises simultaneously bypassing both of the series gates while sealingly engaged by the upstream gate to resist leakage of the fluid out of the simulated bladder.

18. The method of claim 14 wherein filling the simulated bladder using the syringe is performed while the simulated bladder is connected to a support structure.

19. The method of claim 13 further comprising removing the catheter from the one-way valve to stop the release of the fluid from the reservoir.
